(19) United States
(12) Plant Patent Application Publication
(10) Pub. No.: US 2003/0066112 P1
(43) Pub. Date: Apr. 3, 2003

(54) Floribunda rose plant 'POULmax'

(76) Inventors: L. Pernille Olesen, Fredensborg (DK); Mogens N. Olesen, Fredensborg (DK)

Correspondence Address: Poulsen Roser Pacific, Inc., 620 S. Front Street, Central Point, OR, 97502, US

(21) Appl. No.: 10/192,746

(22) Filed: Jul. 9, 2002

Related U.S. Application Data
(63) Continuation of application No. 09/277,238, filed on Mar. 26, 1999, now abandoned.

(51) Int. Cl. A01H 5/00

(52) U.S. Cl. PLT/147

(57) ABSTRACT

A new garden rose plant which has abundant, semi-double salmon pink colored flowers and attractive foliage. This new and distinct variety has shown to be uniform and stable in the resulting generations from asexual propagation.

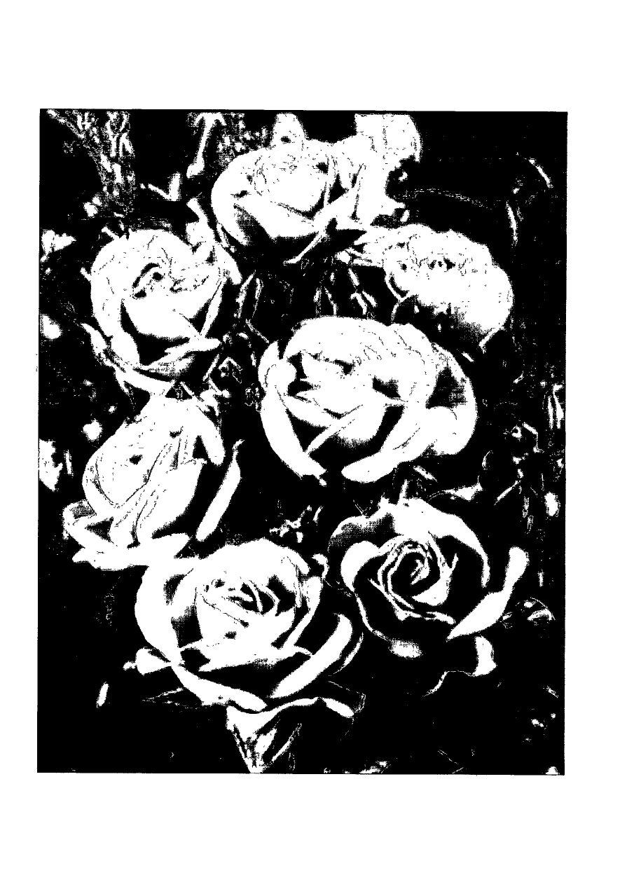

### SUMMARY OF THE INVENTION

The present invention constitutes a new and distinct variety of garden rose plant which originated from a controlled crossing between an unnamed seedling (non-patented) and an unnamed seedling (non-patented). The two parents were crossed in the summer of 1988 and the resulting seeds were planted in a controlled environment. The new variety is named ‘POULmax’. The new rose may be distinguished from its seed parent, an unnamed seedling created by the same inventors, by the following combination of characteristics:

1. The seed parent is a low growing, compact floribunda, while ‘POULmax’ is more upright in habit.

2. The seed parent has soft pink colored flowers, while ‘POULmax’ has salmon pink colored flowers.

The new variety may be distinguished from its pollen parent, an unnamed seedling created by the same inventors, by the following combination of characteristics:

1. The pollen parent is a very compact and bushy floribunda, while ‘POULmax’ is more upright in habit.

2. The pollen parent has soft pink colored flowers, while ‘POULmax’ has salmon pink colored flowers.

3. The pollen parent is not as disease resistant as ‘POULmax’.

The objective of the hybridization of this rose variety for garden use was to create a new and distinct variety with unique qualities, such as:

1. Uniform and abundant flowers;

2. Vigorous, compact growth;

3. Cold hardiness and disease resistance.

This combination of qualities is not present in previously available commercial cultivars of this type and distinguishes ‘POULmax’ from all other varieties of which we are aware.

As part of their rose development program, L. Pernille Olesen and Mogens N. Olesen germinated the seeds from the aforementioned hybridization and conducted evaluations on the resulting seedlings in a controlled environment in Fredensborg, Denmark.

‘POULmax’ was selected by the inventors in the spring of 1989 as a single plant from the progeny of the aforementioned hybridization. Asexual reproduction of ‘POULmax’ by traditional budding was first done by L. Pernille and Mogens N. Olesen in August, 1989. This initial and other subsequent propagations conducted in controlled environments have demonstrated that the characteristics of ‘POULmax’ are true to type and are transmitted from one generation to the next.

### BRIEF DESCRIPTION OF THE DRAWING

The accompanying color illustration shows as true as is reasonably possible to obtain in color photographs of this type, the typical characteristics of the buds, flowers, leaves, and stems of ‘POULmax’. Specifically illustrated in SHEET 1:

1. Stem showing branching and the attachment of leaves, buds, and peduncles;

2. Flower bud, partially opened bud, and open bloom;

3. Flower petals, detached;

4. Sepals, receptacle, and pedicel;

5. Flowering stem as well as a bare stem exhibiting thorns;

6. Leaves.

### DETAILED DESCRIPTION OF THE VARIETY

The following is a description of ‘POULmax’, as observed in its outdoor growth in a field nursery in Jackson County, Oregon, on plants aged eighteen months. Observations were conducted during October, 1998. Color references are made using The Royal Horticultural Society (London, England) Colour Chart, 1995, except where common terms of color are used. For a comparison, several physical characteristics of the rose variety ‘POULander’, a rose variety from the same inventors described and illustrated in U.S. Plant Pat. No. 6,265 and issued on Aug. 30,1988 are compared to ‘POULmax’ in Chart 1.

CHART 1

| | ’POULmax’ | ’POULander’ |
|---|---|---|
| Flower bud color, | Red Group 46C-47D. | Red Group 46A. |
| as sepals divide to | | |
| ¼ open. | | |
| Color, upper | Red Group 43D. | Red Group 46B TO |
| petal surface, | | 46C. |
| upon opening. | | |
| Petalage under | 18-22 petals. | Approx. 20 petals. |
| normal | | |
| conditions. | | |

Parents:

Seed parent.—An unnamed seedling.

Pollen parent.—An unnamed seedling.

Classification:

Botanical.—Rosa hybrida.

Commercial.—Floribunda.

### FLOWER AND FLOWER BUD

Blooming habit: Continuous.

Flower bud:

Size.—Upon opening, 33 mm in length from base of receptacle to end of bud.

Bud form.—Medium, pointed ovoid.

Bud color.—As sepals unfold, Red Group 46 C. Red Group 47 D at ¼ opening.

Sepals.—Inner side Yellow-Green Group 146 A, outer side Yellow-Green Group 146 C. Foliaceous appendages lacking. Surfaces of sepals slightly pubescent. Limited numbers of stipitate glands present on margin of sepals. Sepals are 25 to 30 cm long and 7 to 10 cm wide. Apex is subulate in shape.

Receptacle.—Surface: Pubescence very strong. Shape: Funnel. Size: Medium, 7 mm (h)×8 mm (w). Color: Green Group 143 C.

Peduncle.—Surface: Slight pubescence. Stipitate glands lacking. Length: 50 mm to 70 mm. average length. Color: Yellow-Green Group 144 A. Strength: Strong.

Borne.—With 4-5 buds per flowering stem.

Flower bloom:

Fragrance.—Light floral scent.

Duration.—As a cut flower 4 to 5 days. The blooms have a duration on the plant of approximately 3 to 7 days. Petals fall cleanly away from plant.

Size.—Average flower diameter is 70 mm when open.

Form.—Semi-double. Shape of flower when viewed from the side: Upon opening, upper part: Flat. Upon opening, lower part: Flat. Open flower, upper part: Flattened convex. Open flower, lower part: Concave.

Petalage.—Semi-double. Average range: 18-22 petals under normal conditions. Generally with 1-2 petaloids.

Color:

Upon opening, petals.—Outermost petals: Upper Surface: Red Group 43 D. Reverse Side: Red Group 48 C-48 D. Innermost petals: Upper Surface: Red Group 43 D. Reverse Side: Red Group 43 D.

Upon opening, basal petal spots.—Outermost petals: Outer Side: Green-Yellow Group 1 C. Inner Side: Yellow Group 2 B. Innermost petals: Outer Side: Yellow Group 2 A. Inner Side: Yellow Group 6 A.

After opening, petals.—Outermost petals: Upper Surface: Red Group 50 C. Reverse Side: Red-Purple Group 58 D. Innermost petals: Upper Surface: Red Group 48 C. Reverse Side: Red Group 52 D.

After opening. basal petal spots.—Outermost petals: Outer Side: Green-Yellow Group 1 D. Inner Side: Green-Yellow Group 1 B.

Innermost petals.—Outer Side: White Group 155 D. Inner Side: White Group 155 D Note: On some open blooms, a white variegation is present in the center of the petal on some of the inner petals.

General Tonality: On the open flower, Red Group 48 C. No change in the general tonality at the end of the 3rd day. Afterwards, general tonality is Red Group 49 B.

Petals:

Petal Reflex.—Slight reflex.

Petal Edge.—Entire.

Shape.—Apex is mucronate; whole petal is rounded deltoid.

Petaloids.—2 to 3. Petaloids are 8 to 10 mm long and 3 to 5 mm wide. Coloration is Red Group 43 C to 43 D. Texture is smooth.

Texture.—Smooth.

Arrangement.—Not imbricated.

Reproductive Organs:

Pistils.—Quantity: 25 to 30. Length: 7 to 10 mm.

Pollen.—Color: Greyed-Orange Group 163 A. Quantity: Above average quantity of pollen.

Anthers.—Size: 2 mm long. Color: Yellow-Orange Group 22 C. Quantity: 30 to 40.

Filaments.—Color: Yellow-Green Group 153 D at the base, changing to Green Group 18 A toward the anthers. Length: 4 to 5 mm.

Stigmas.—Slightly superior relative to anthers. Color: Yellow-Green Group 145 D.

Styles.—Color: Yellow-Green Group 145 B.

Hips.—None observed.

### PLANT

Plant growth: Moderately vigorous and bushy. After two seasons, the average height of a budded field grown plant on R. multiflora understock is 110 cm and the average width is 75 cm.

Stems:

Color.—Young wood: Yellow-Green Group 146 B. Older wood: Yellow-green Group 148 B.

Thorns.—Incidence: 7 to 10 per 10 cm of stem. Size: Average length: 10 mm. Color: Yellow-Green Group 151 A. Shape: Slightly concave.

Surface.—Young wood: Smooth. Older wood: Smooth.

Plant foliage: Quantity of leaflets on leaves in middle of the stem: 5 leaflets, sometimes 7 leaflets.

Leaf size.—Medium 11 mm (1)×7.5 mm (w).

Abundance.—Average.

Color.—Upper Leaf Surface: Yellow-Green Group 147 A. Lower Leaf Surface: Yellow-Green Group 147 B. Juvenile foliage: Yellow-Green Group 147 B. Anthocyanin intonation: Yes. Location: Sepals, receptacle, peduncle, thorns, leaves (especially veins in leaflets), and younger stems. Color: Greyed-Red Group 181 A.

Plant leaves and leaflets:

Stipules.—Size: 7 mm (1)-4 mm (w). Color: Yellow-Green Group 147 B. Presence of stipitate glands: On upper portion of stipules and on stipule margins. Color is unusual, near Violet Group 86 A.

Petiole.—Length: 20 mm-30 mm. Color: Yellow-Green Group 147 B. Underneath: With small prickles. Stipitate glands lacking. Margins: Stipitate glands in very limited numbers along margins and also on leaflet petiole.

Rachis.—Color: Yellow-Green Group 147 B. Underneath: Color Yellow-Green Group 147 B. With small prickles. Margins: Stipitate glands present in limited numbers.

Leaflet.—Edge: Serrated. Shape: Ovate. The leaflet's apex is acuminate. The leaflet's base is rounded. Arrangement: The leaflets are arranged in an odd-pinnate formation. Venation: The leaflets are veined in a reticulate pattern. Texture: Upper side of leaflet is moderately glossy. Lower side of leaflet is matte.

Disease resistance: Exceptional resistance to rust, Black spot, and Botrytis under normal growing conditions in Jackson County, Oregon.

Winter hardiness: ‘POULmax’ has been found to be resistant to damage from cold in USDA Zone 8 and USDA Zone 7.

1. A new and distinct variety of rose plant of the floribunda class, substantially as herein illustrated and described as a distinct and novel rose variety due to its abundant, salmon pink flowers, vigorous growth, disease resistance, and extended period of bloom:
